(19) United States
(12) Patent Application Publication
(10) Pub. No.: US 2012/0053661 A1
(43) Pub. Date: Mar. 1, 2012

(54) WEARABLE, MOTION ACTIVATED BODY PART WARMING DEVICE

(76) Inventor: Gary Neil Hooper, Thornhill (CA)

(21) Appl. No.: 13/216,899

(22) Filed: Aug. 24, 2011

Related U.S. Application Data
(60) Provisional application No. 61/376,812, filed on Aug. 25, 2010.

(51) Int. Cl. A61F 7/08

(52) U.S. Cl. 607/104

(57) ABSTRACT

A wearable device configured as an article of apparel is used for warming a body part having a core such as a hand palm and an extremity such as a finger The wearable device has a circulatory system containing a heat transfer fluid, tubes located next to the extremity for heat transfer from the tubes to the extremity, and chamber located next to the core for heat transfer from the core to the fluid in the chamber. The circulatory system has a pump operable in response to movement of the body part to pump the heat transfer fluid around the circulatory system.

### CROSS REFERENCE TO RELATED PATENTS

This patent application is claiming priority under 35 USC §119 to a provisionally filed patent application entitled WEARABLE MOTION ACTIVATED BODY HEAT TRANSFER DEVICE, having a provisional filing date of Aug. 25, 2010 and a provisional serial number of 61,376,812.

### FIELD OF THE INVENTION

This invention relates generally to wearable items such as boots, shoes, gloves and the like which are capable of transferring body heat to a body part extremity.

### DESCRIPTION OF RELATED ART

People operating machinery, working and otherwise performing activities in cold environments often experience discomfort and numbness in various body extremities. For example, hunters, fishermen, military personnel, explorers, sportsmen and outdoor workers, who are exposed to long or intense periods of cold weather, often experience numbness and discomfort in body parts such as hands and feet, especially the extremities such as fingers and toes. In order to minimize the discomfort and numbness, insulated gloves may be worn. However, in particularly cold weather, even insulated gloves have a tendency to let fingers become numb due to the loss of heat through the fingers of the glove. Any body extremity, such as the toes of the feet, can experience numbness and cold when the wearer is exposed to cold weather for long periods of time. Insulated boots and gloves, while offering a degree of protection, are often not very effective because circulation to the toes and fingers is reduced as the human body experiences cold. Several attempts have been made to overcome this problem by providing electrically heated gloves and boots. These electrically heated devices use electric storage batteries to operate electric resistance heaters located in the fingers of the glove or the toes compartment of the boot (as the case may be). While effective, these electrically operated devices can only operate for a relatively short time before requiring either new batteries or a recharging of the batteries, and they may require that an individual carry extra weight or wear a bulky battery belt or similar apparatus. Therefore an improved wearable device which permits the extremities of the body to remain warm without requiring electrical currents is therefore required.

### SUMMARY OF THE INVENTION

According to one aspect of the invention, there is provided a wearable device for warming a body part of a wearer, the body part having a core part and an extremity part, the wearable device comprising a circulatory system containing a heat transfer fluid, the circulatory system having a first heat transfer element for wearing adjacent the body part extremity for heat transfer from the first heat transfer element to the extremity, and a second heat transfer element for wearing adjacent the body part core for heat transfer from the core to the second heat transfer element, the circulatory system including a pump operable in response to movement of the body part to pump the heat transfer fluid around the circulatory system. The pump can be operable in response to a movement of the body part that is one of (a) an articulating movement, (b) a movement relative to a second body part and (c) a movement involving contact with a reference surface not part of the body.

The wearable device can be contained within a housing of an insulating fabric such as a glove, boot or other article of apparel or within an insulating liner for such an article. Alternatively, the wearable device can be an integral part of such a housing. The pump preferably includes a first chamber having a flexible, relatively inelastic wall material, a second chamber having a flexible relatively elastic wall material, and a passage containing a valve for one-way flow of fluid from the second chamber to the first chamber. The circulatory system can further include a plurality of tubes, the tubes having valves permitting only one-way flow of heat transfer fluid through the tubes, each tube linking the first to the second chamber, being in fluid communication therewith, and being of a length to extend from the chambers to an extremity of the body part and back. Preferably, the valves are duckbill valves formed integrally with and internal to the tubes.

For a hand body part, the insulating housing can be a glove, the pump operable by the wearer making grasping and releasing movements or making some other finger or hand movement. The movement can be a deliberate movement implemented for the purpose of pumping heat transfer fluid around the circulatory system. Alternatively, the pump can be operable as a side effect of the wearer taking some other action for another purpose, such as grasping an object, turning a wrench, hauling on a rope or cable, etc. The core part can be a hand palm with the second heat transfer element located next to the palm when the device is worn. The extremity part can be a finger with the first heat transfer element located next to or surrounding the finger when the device is worn.

For a foot body part, the insulating housing can be a boot, the pump operable by the wearer walking or otherwise effecting movement of active parts of the foot such as the heel or toes. The core can be the foot's sole with the second heat transfer element located next to the sole when the device is worn. The extremity can be a toe with the first heat transfer element located next to the toe when the device is worn.

In such a wearable device, the tubes of the circulatory system can have a range of bore sizes and shapes. The tubes of the circulatory system can branch towards the extremity, the branches combining towards the core.

The heat transfer fluid preferably has a freezing point below the freezing point of water and may be a solution of ethylene glycol in water.

According to another aspect of the invention, there is provided a method of warming a body part using a device worn at the body part, the body part having a core part and an extremity part, the method comprising moving the body part so as to operate a pump forming part of the device to pump heat transfer fluid through a circulatory system forming part of the device, transferring heat from a first heat transfer element forming part of the device to the extremity part, and transferring heat from the core part to a second heat transfer element forming part of the device.

### BRIEF DESCRIPTION OF THE DRAWINGS

For simplicity and clarity of illustration, elements illustrated in the following figures are not drawn to common scale. For example, the dimensions of some of the elements are exaggerated relative to other elements for clarity. Advantages, features and characteristics of the present invention, as well as methods, operation and functions of related elements of structure, and the combinations of parts and economies of manufacture, will become apparent upon consideration of the following description and claims with reference to the accompanying drawings, all of which form a part of the specification, wherein like reference numerals designate corresponding parts in the various figures, and wherein:

FIG. 1 is a view from above showing the interior of a glove in accordance with one embodiment of the invention.

FIG. 2 is a view from the side showing a hand and elements of a wearable device in accordance with an embodiment of the invention, the hand being shown in an open position.

FIG. 3 is a view corresponding to FIG. 2 but showing the hand in a closed position.

FIG. 4 is a sectional view showing a wall between an output and intake chambers forming parts of a glove according to another embodiment of the invention.

FIG. 5 shows one form of polythene tube configured in the form it occupies in a glove according to an embodiment of the invention.

FIG. 6 is a schematic representation of a branching polythene tube formation for use in a glove according to an embodiment of the invention.

FIG. 7 shows a wearable device according to an embodiment of the invention, the device configured as a liner device for insertion by a wearer into an insulating glove housing.

FIG. 8 is a view from one side showing the interior of a boot embodying a wearable device in accordance with an embodiment of the invention.

FIG. 9 is a view from below showing the boot of FIG. 8.

FIG. 10 shows a view from one side showing the interior of a glove in accordance with an embodiment of the invention, the glove being shown with the gloved hand in successive configurations.

FIG. 11 shows a schematic view of tubing for us in a wearable device in accordance with an embodiment of the invention.

### DETAILED DESCRIPTION OF THE INVENTION INCLUDING THE PRESENTLY

### Preferred Embodiments

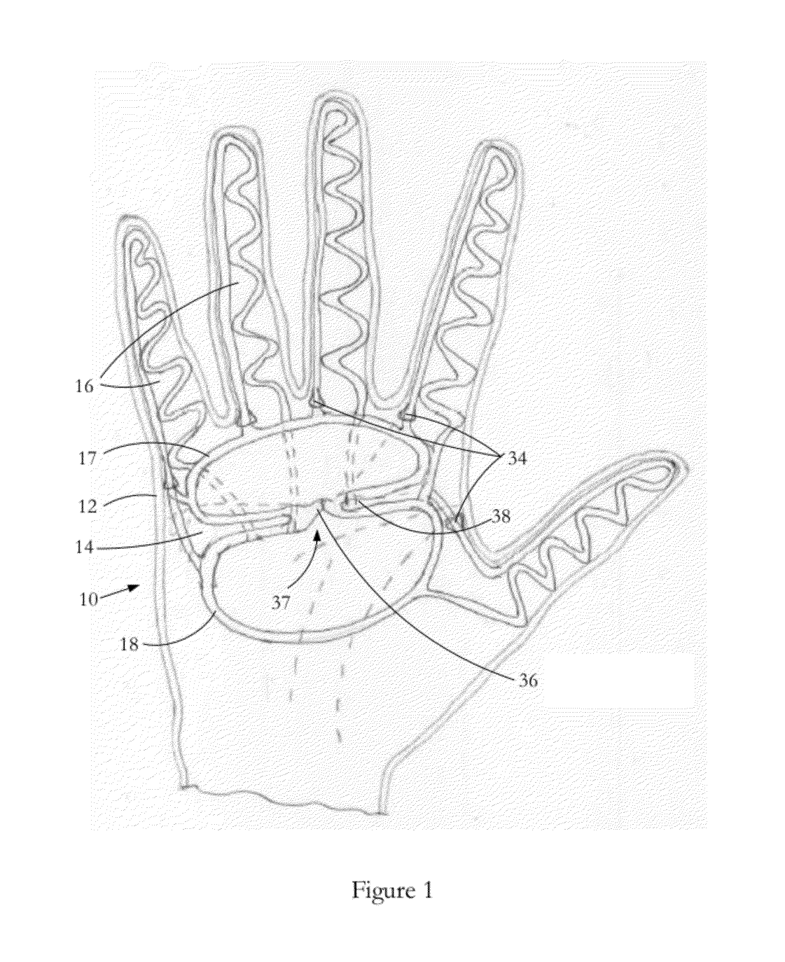

Referring in detail to FIG. 1, a glove made in accordance with the present invention is shown generally at 10 and includes a flexible insulating glove housing 12 having a palm portion 14 and finger portions 16 which are configured respectively to receive the palm portion and fingers of a wearer's hand. The glove housing 12 is made from a flexible textile material which provides protection from the cold and which has good wear resistance. Suitable multi-layer fabric designs which incorporate protective outer fabric layers and internal insulation layers are readily available in the marketplace. The glove housing 12 is sufficiently flexible to permit the wearer easily to open and close the gloved hand and to flex the fingers.

Contained within the glove housing 12 are flexible containers or chambers 17, 18 and tubes 20. The chamber 17 is an “output” chamber made of a relatively inelastic wall material, whereas the chamber 18 is an “intake” chamber made of a relatively elastic wall material. Each of the chambers contains a volume of heat transfer fluid 21 which, in the embodiment shown, is a mixture of water and ethylene glycol, the mixture having a freezing point below that of water. However, any liquid compatible with the material forming the chambers 17, 18 and tubes 20 may be used depending on the heat transfer and other properties sought.

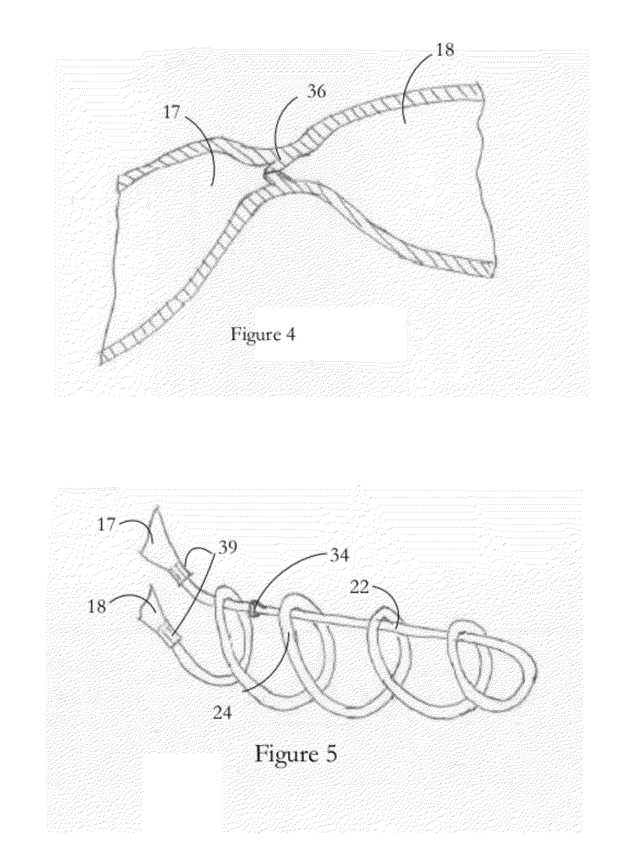

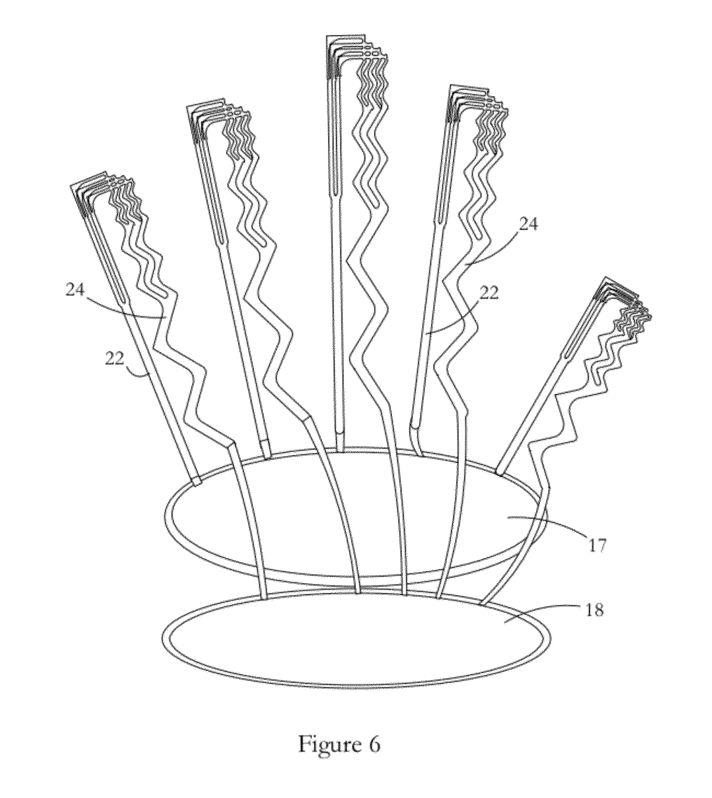

As shown in the sectional view of FIG. 4, a fluid passage 37 is formed in a seam 38 between the chambers 17, 18, with the passage housing a one-way check valve 36 to permit the heat transfer fluid 21 to pass in one direction from the intake chamber 18 to the output chamber 17, but not in the reverse direction. For simplicity of construction, the check valve is configured as a duckbill valve, so called because it is characterized by an elastic operational element shaped as a duck's bill. The material and dimensions of the elastic element and its surrounding support material are selected so that the elastic element deforms as a result of a predetermined fluid pressure gradient applied across the elastic element. When pressure is applied in one direction by means of pumping fluid from a high pressure area, the valve opens. When the pressure gradient is removed or pressure is applied in the opposite direction, the valve closes as the elastic element reverts to its rest condition and shape. As shown in the representation of a single one of the tubes 20 as shown in FIG. 5, the tubes have portions 22 whose ends are in fluid communication with the output chamber 17 and which extend away from chamber up through respective finger portions. The tubes 20 have portions 24 whose ends are in fluid communication with the chamber 18 and which extend down along the respective finger portions into the chamber 18. Each tube 20 has at least one check valve 34 which restricts the liquid to flow one way from the output chamber around the tubes system to the intake chamber. For simplicity of manufacture, the check valves 34 are also configured as duckbill valves. The tubes 20 are made of extruded silicone, polyethylene, polyvinyl or other synthetic material, the properties of which are chosen for manufacturability and for the conditions to which the glove is expected to be subjected. The tubes have an internal bore size typically in the range 0.5 to 4 millimetres. In one embodiment of the invention, lengths of the tubes 20 are sealed to duckbill valves by suitable sealant matched to the material of the tube and the valve. In another embodiment, polyethylene tubing is extruded in a process which produces internal one-way duckbill valve formations at periodic positions along the extrusion. The tubes 20 are also sealed to the chambers at entry and exit flanges 39. The material of the tube, the chambers, the valves and all joint regions is made resilient to combat rough usage of the gloves and other expected environmental conditions such as temperature and moisture. In the tube formation of FIG. 5, the “go” portions 22 are relatively straight while the “return” portions 24 are coiled. In this way, warmed fluid is transported directly to the finger tips so as to lose only a minimal amount of heat by the time the fluid reaches the finger tips which are normally the finger parts most prone to exposure and reduced circulation. However, a reverse formation can be adopted with the warmed fluid passing through the coiled part of the tube before reaching the finger tips and then being conveyed relatively directly back to the chamber 18. Variations on such an arrangement are possible. For example, multiple separate subsidiary circuits can be used to direct warmed fluid to different regions of a particular finger.

The chambers 17, 18 are positioned in the glove housing 12 so as to be in close thermal contact with the palm of a person wearing the glove. Similarly, the tubes 20 lie in close thermal contact with the user's fingers when the glove is being worn. As shown in FIGS. 1 and 2, the chamber 17 lies adjacent a region of the wearer's palm towards the wearer's fingers, and the chamber 18 lies adjacent a region of the wearer's palm towards the wearer's wrist. The dual chamber structure is positioned within the glove housing 12 such that the seam 38 is at or close to a natural fold line where the palm portion of the glove housing 12 is folded when the wearer closes the hand.

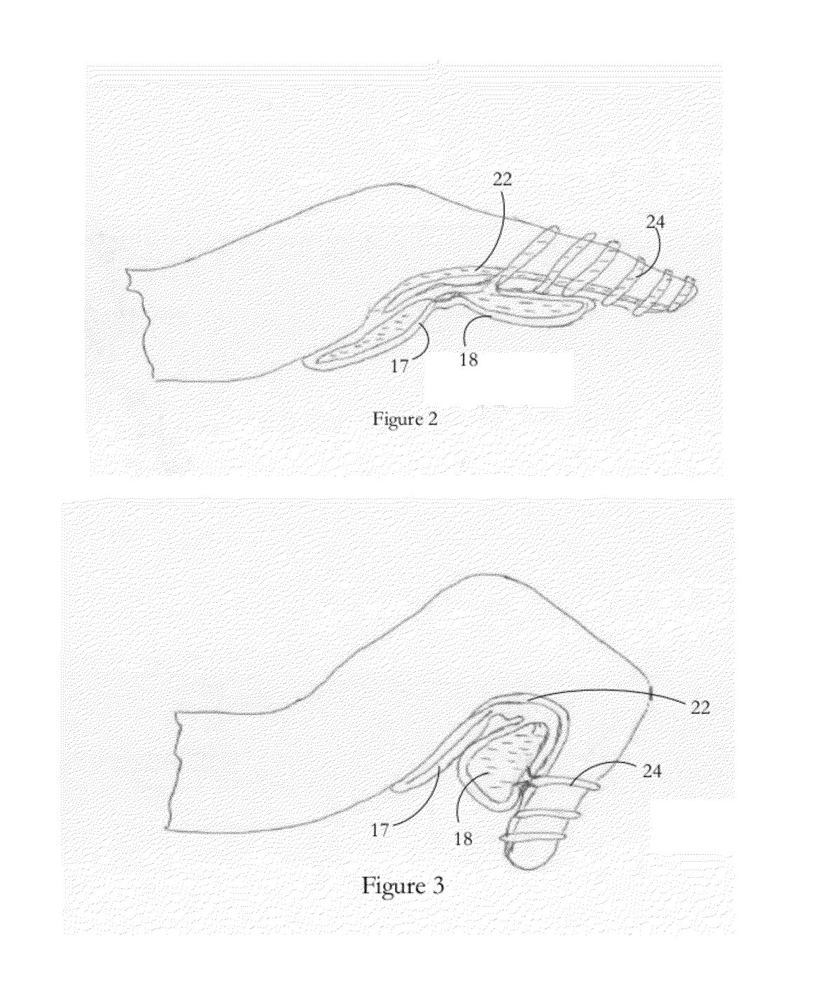

The chambers 17, 18 and the tubes 20 are configured as a circulatory system having a simple pump for driving the heat transfer fluid 21 through the chambers 17, 18 and the tubes 20. The pump action is generated at the linked chambers 17, 18 by the glove wearer lightly clenching and unclenching a fist, the unclenched and clenched positions being shown in FIGS. 2 and 3 respectively. Other similar hand movement may be used to actuate the pump. These may be actions deliberately taken by the glove wearer to stimulate circulation of the heat transfer fluid or may occur as a side effect of an action intended for another purpose such as manipulating a ski pole or turning a wrench. When the fist is clenched, the chamber 17 is squeezed and its volume is decreased causing heat transfer fluid 21 to be driven out of the chamber into the tubes 20. The fluid is prevented from flowing directly from the chamber 17 into the chamber 18 through the passage 37 because, in response to increasing fluid pressure from the chamber 17, the check valve 36 is prevented from opening. The fluid is, however, driven into the tube portions 22, along the fingers to the finger extremities, and then back down the fingers through the tube portions 24 and into the chamber 18. Because the chamber 18 is made of an elastic material, it expands to accommodate the fluid returning from the finger portions so that the heat transfer fluid becomes distributed between the chambers 17 and 18 as shown in FIG. 3.

Subsequently, when the chamber 17 is released by unclenching the fist, heat transfer fluid 21 flows through the passage 37 and the one-way valve 36 from the chamber 18 to the chamber 17 to restore the chamber 17 to the start position shown in FIG. 2. The fluid is driven from the chamber 18 to the chamber 17 by a sucking action as the chamber 17, owing to its inherent resilience, reverts to its unsqueezed shape, and by a driving action as the expanded chamber 18, owing to its inherent elasticity, deflates to its start shape and volume. While the heat transfer fluid is both drawn and driven as the respective chambers 17 and 18 revert to their start conditions, the fluid 21 may alternatively be predominantly or solely moved by one or other of these mechanisms.

In the illustrated embodiment, the chambers 17, 18 are separate chambers with a seam 38 uniting them and with the passage 37 housing the check valve 36 contained in the seam 38. The chambers 17, 18 may however be separate containers having linking tubes, or may form a dual chamber structure having a dividing wall separating the two chambers and with a passage and check valve formed or mounted in the wall. Communication between the two chambers may be by a single or multiple passages 37 housing respective check valves 36. In the embodiment shown, the double chamber structure has a shape and size not unlike a conventional cushioning insole.

Whereas each of the tubes 20 in the embodiment illustrated in FIG. 1 has a circular bore of a standard internal and external bore size and a standard wall thickness, other embodiments may have tubes or tube portions differing in bore and wall size from other tubes or tube portions depending on the position and function of the particular tube or tube portions in the circulatory system. The tubes 20, or at least a portion of them, may be non-circular in form. For example, a flattened profile at the finger extremities may encourage the tubes to lie flat against the fingers and so offer a greater contact surface for heat transfer than would a conventional circular bore tube. Such a profile may also be adopted in tubes or portions thereof where a circular profile may be more likely to cause irritation to the wearer. On the other hand, tubes or portion thereof which are located at places where they may be subject to considerable physical wear, such as certain locations of a work glove, are formed with circular profile so as to be less prone to damage.

The size and other characteristics of the tubes 20 and other elements such as the valves 34 may also be selected for a desired appearance of the glove. For example, small internal bore tube of the order of 0.5 to 4 millimetres is used if a device of small profile is desired and if it is acceptable in the expected glove usage to require frequent fist pumping. On the other hand, larger bores and valves are used if a bulky appearance is unimportant and it is desired to have appreciable circulation of fluid in response to a fairly low frequency fist clenching or other hand movement to effect the pump action.

Particularly for an arrangement in which the tubes 20 are integrated with a glove housing material, the tubes 20 may have a branching structure so that, for example, as the tube portions 22 extend away from the chamber 17, they branch into smaller tubes. As shown in the schematic representation of FIG. 6, the branching may be progressive so as to culminate in a lattice of capillary tubes at the finger tips so giving a relatively large contact area for heat transfer. In a similar way, the return portions of such a branched tube structure progressively converge towards the intake chamber 18. Such a structure is analogous in some respects to a respiratory system and has an analogous purpose. In an alternative configuration, the “go” tube portions 22 are relatively wide bore with a single tubular portion 22 extending relatively directly along each finger while the “return” tube portions 24 are branching and follow more circuitous paths as they return to the chamber 18. While the branching into smaller capillary tubes is shown at the finger tips, the branching may occur at any position along the finger if greater thermal contact area is desired at such a position.

As previously noted, the chambers 17 and 18 are made from different wall materials or material compounds to obtain the desired elasticity and other properties required for permitting the fluid pumping action. To optimize the two functions of the dual chamber structure—the heat sink function and the pumping function—the wall material of one or both of the output and intake chambers may vary over the area of the wall.

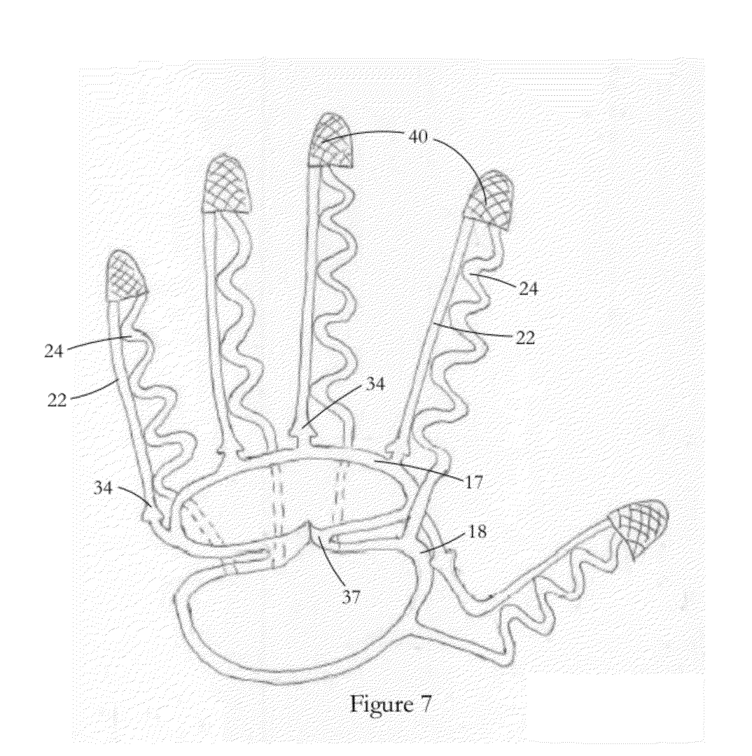

The embodiment of FIG. 1 shows a warming arrangement in which elements such as the tubes 20 are bonded to or woven into parts of the insulating material of a glove. In accordance with another embodiment of the invention as shown in FIG. 7, a separate device is strapped onto the wearer's hand before the wearer inserts the hand into a conventional, relatively loose-fitting glove. The device has the same elements as are illustrated in the FIG. 1 embodiment, but without the surrounding insulating glove housing 12. Thus, the arrangement has flexible chambers 17, 18 which are mounted on an elastic strap into which the wearer's hand can be inserted. Extending from the chambers is a lattice of tubes 20 with tubes for specific fingers terminating at wring- or thimble-type mounting members 40. The tubes 20 have the same arrangement of go and return tubular portions as illustrated in the FIG. 1 embodiment. In use, the wearer dons the device by placing the hand through the strap 42 with the double chamber against the wearer's palm and putting finger tips into respective wrings or thimbles 44 so that associated tubes 20 are arranged along respective fingers. The hand, with device attached, is then slid into a conventional loose-fitting glove. The device is actuated in the same way as the integral device of FIG. 1 by clenching and unclenching the fist to drive the heat transfer fluid through the circulatory system.

As shown in FIG. 1, the chamber 17 is positioned in direct physical contact with the hand palm to maximize the efficiency of heat pick-up from the palm of the hand to the liquid contained in the chamber. To maximize heat transfer, the chamber 17 can have its contact surface pre-moulded to the surface contouring of the particular wearer's palm as configured when the wearer's palm is in an unclenched position and the chamber 17 is in its open, unsqueezed condition. The chamber 17 may alternatively be separated from the palm of the wearer's hand by no more than a thin layer of flexible material. While little or no insulation separates the chamber 17 from the wearer's hand, the glove housing 12 consists of or includes a layer of insulation surrounding the chambers 17 and 18 to prevent loss of heat from the glove.

As previously indicated, a preferred heat transfer fluid 21 is a solution of ethylene glycol and water because such a solution has both the desired low temperature behaviour and a reasonable specific heat capacity from the viewpoint of absorbing and transferring heat. However, the application of the invention contemplates other fluids.

In terms of heat pick-up, the chamber 17 is the primary element both because it is located directly adjacent the warm palm when the device is worn, and because it is from this chamber that the heat transfer fluid is driven into the tubes 20. A supporting role is played by the fluid in the chamber 18 because it too is located next to the warm palm and because a part of its contents are flushed through to the chamber 17 during each fist clenching cycle. In use, the chambers 17, 18 and tubes 20 effectively cause glove finger portions 16 to be warmed by fluid circulating around the circulatory system consisting of the chambers and tubes. In this way, heat is taken from the wearer's palm, which is generally fleshy, well supplied with blood vessels, and therefore relatively warm, to the wearer's fingers, which are more susceptible to cold. This heat transfer occurs naturally as the wearer goes about his or her activities while wearing the glove. The natural action of flexing the hand, and the more pronounced action of opening and closing the fist, causes the pumping mechanism to transfer heat from the warm palms to the cold fingers, thereby reducing the chance of the wearer's fingers becoming numb.

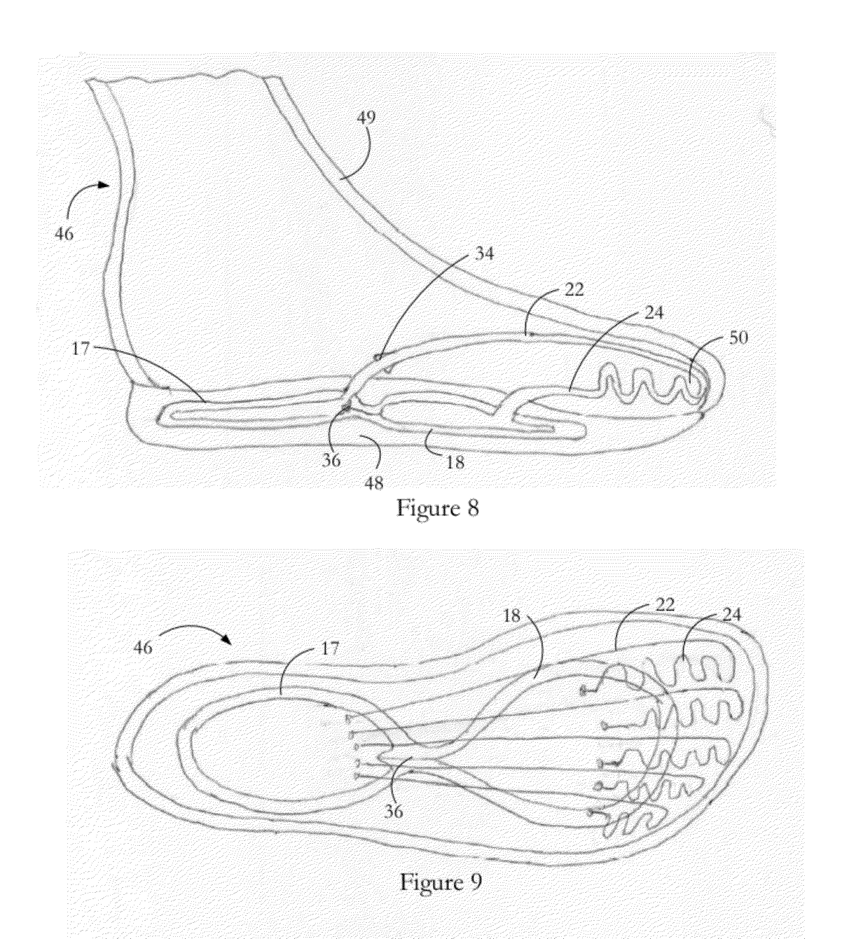

As shown in FIGS. 8 and 9, the structure and operation of a warming device according to the present invention can alternatively be incorporated in a boot or shoe shown generally at 46. In such an embodiment, an insulating boot housing 49 includes conventional outer elements of a boot including a sole portion 48 and a toe portion 50. A structure comprising output and intake chambers 17, 18 is positioned in the sole of the boot, so that when the boot is worn, the dual chamber are supported by the sole of the boot, but are in close thermal contact with the wearer's foot arch. Tubes 20 connected to the chambers 17 and 18 extend into the toe portion of the boot and have go and return portions, 22 and 24 respectively, for the transfer of warmed fluid towards the toes and for return of cooled fluid from the toes to the heat pick-up zone. The tubes 20 are shown as lines rather than tubes in FIG. 9 in the interests of clarity. In addition, it will be appreciated that in a practical implementation, the tubes 20 occupy paths somewhat different from those shown in FIGS. 8 and 9 in order comfortably to accommodate the wearer's foot. The pump action is brought about by the fluid filled chamber 17 being successively squeezed and released and the chamber 18 being successively expanded and deflated. The relatively inelastic chamber 17 is squeezed as the wearer brings the heel down against the ground during the action of walking, running, etc. The chamber 17 is released as the foot is lifted and the expanded elastic chamber directs fluid that has returned from the toes into the chamber 17. Fluid flow from the chamber 18 to the chamber 17 is aided by the wearer's pressure point with the ground moving from the heel to the ball of the foot. This pump action may be supplemented by some articulation of the bones of the foot. In this case, the motive force for driving the heat transfer fluid around the circulatory system is somewhat different from that of the glove embodiment. As in the case of the glove, the heat transfer fluid circulates around the circulatory system comprised of the chambers and the tubes, picking up heat at the sole region and releasing the heat at the toes. Heat is transferred from the sole of the wearer's foot to the toe portion of the boot to reduce the chance of the wearer's toes from becoming painful or freezing.

While embodiments of the invention have been described in which the heat transfer chambers are designed to be either against the hand palm in the case of the glove and against the sole of the foot in the case of a boot or shoe, it will be appreciated that the principles of the invention can be realized by positioning the chambers (or other heat transfer element) against any region of a body part which is normally warmer than another region of that body part so that a heat gradient between the two body part regions exists. Hands and feet are the body parts where the invention is considered to have most application, however, because fingers and toes are especially prone to the cold as they are somewhat exposed even with a good glove or boot covering, and the circulation to them is somewhat spare because they are at the outer zones of the body's blood circulation system. While other body parts may also offer a site where there is a temperature gradient that can be utilized for heat transfer purposes, it is important that the heat pick-up, such as the hand palm, be close to the heat sink, such as the fingers, because otherwise a significant portion of the heat picked up may be lost in transit before it can be conveyed to the extremities, such as fingertips, where it is most needed. Generally, depending on the particular body part whose extremity(ies) is (are) to be warmed, the pump drive may utilize any or all of (a) articulation of the elements of a body part relative to each other, (b) articulation of the body part relative to another body part, (c) movement of the body part relative to a reference surface such as the ground and (d) translational or twisting movement of the body part occasioning local changes of momentum in the heat transfer fluid.

In the embodiment of the invention illustrated in FIG. 1, the pump is at the same general location as the heat pick-up. Pump mechanisms have designs and operating principles different from the example illustrated and described are envisioned provided that the pump operational elements are actuated by movement of the hand, foot or other body part sufficiently for the heat transfer fluid to be driven through the circulatory system from the warm or core region to the colder extremity region.

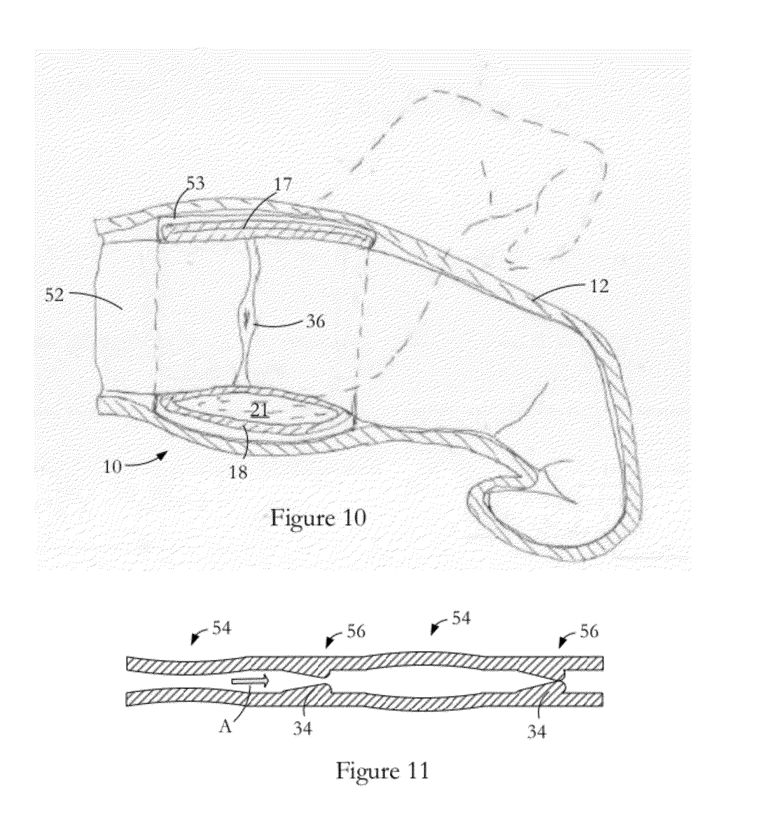

It will be appreciated that to optimize heat pick-up and pump action, it may be necessary or convenient to locate operational elements of the pump at a location different from the location of operational elements of the heat pick-up. For example, as shown in FIG. 10, in an alternative embodiment, a pump action chamber arrangement is located adjacent the wearer's wrist 52 and is actuated by cocking and uncocking the wrist. The output and input chambers are located above and below the wrist and are surrounding by an elastic wristband 53. In the uncocked position (solid line), a lower inelastic chamber—the output chamber 17—contains a volume of heat transfer fluid. When the wrist is cocked (broken line), the elastic band squeezes the chamber 17 to drive fluid 21 through passage and check valve into the elastic wall chamber 18 which has been opened up by the cocking of the wrist to move the wrist band away from a central regions of the chamber 18. The circulatory system in other respects is similar to that described with reference to the FIG. 1 embodiment and the heat sink chamber (not shown) is similarly located at the user's palm. In a further embodiment, not shown, a plurality of linked output and/or intake chambers can be used to harness to the pump action the many natural movements that occur in the use of a hand, foot or other body part as the case may be. Similarly, whereas it is convenient to have one or two core heat transfer element such as the fluid in chambers 17 and 18, a larger number of linked heat transfer elements can be used, these being disposed to pick up heat from whichever body part regions will normally have a higher temperature in relation to other regions to be warmed.

In a further embodiment as shown in FIG. 11, the pump elements are distributed throughout the circulatory system. In such an arrangement, the tubes 20 are microtubes having valve zones 54 and transfer zones 56. The valve zones 54 house duckbill valves enabling heat transfer fluid flow around the circulation system in a desired circulatory direction as shown by arrow A but preventing any such flow in a reverse direction. In the transfer zones, the polythene tube has a wall thickness and resilience such that, through movement of adjacent regions of the hand and corresponding flexure of the tubes at the transfer zones, the volume of fluid that can be retained in each transfer zone oscillates. As the volume reduces, fluid tends to be driven out of the transfer zone. Because fluid cannot be expelled upstream through the check valves, it is expelled downstream. Consequently, with repeated tube flexure through all or part of the tube system, there is a general migration of the thermal transfer fluid around the circulatory system. This distributed pump system may be the sole agent of fluid transfer or may be an adjunct to a localized pump such as that previously described.

In manufacturing a glove incorporating the present invention, normal glove-making production processes are modified. In one such method, the chambers and tube elements are assembled and brought to the glove while glove panels are still open and before they have been stitched or bonded together. An adhesive is then used to stick the elements of the glove warming device in the desired positions against the exposed glove panels. In an alternative method for manufacturing gloves with the warming device, the tubes are woven or sealed along desired paths into or onto glove panel material so as to leave tubular pigtails for connection to the chambers. The glove panel material is then cut into panels, the tube pigtails are sealed at exit and entry flanges formed in the walls of the chambers. Following injection of the heat transfer fluid into the circulatory system, the panels, with the chambers bonded in place by adhesive, are finally assembled and bonded or stitched into the finished glove.

The present invention has advantages over the prior art. In particular, since the system of chambers and tubes forms a circulating pump which is operated by the wearer during the normal use of the article, no batteries or consumables are needed. Hence, a pair of gloves or a pair of boots incorporating the heat transfer system of the present invention does not require maintenance. This permits the boots or gloves to simply be picked up and used just like any other pair of boots or gloves. From the wearer's perspective, the only noticeable difference is that the glove's finger portions or the boot's toe portions (as the case may be) do not get as cold as they otherwise might.

Embodiments of the invention have been described in relation to gloves and boots. It will be appreciated that the invention is equally applicable to mitts, shoes and other items that can be worn on a body part and where the invention can be implemented with a perceptible warming effect from one region of the body part to another region of the body part. Any reference in the specification to gloves shall encompass mitts and like apparel and any reference to boots shall encompass shoes and like apparel.

Specific embodiments of the invention have been disclosed. However, several variations of the disclosed embodiment could be envisioned as within the scope of this invention. It is understood that the present invention is not limited to the embodiments described above, but encompass any and all embodiments within the scope of the following claims. Other variations and modifications will be apparent to those skilled in the art. The embodiments of the invention described and illustrated are not intended to be limiting. The principles of the invention contemplate alternatives having advantages and properties evident in the exemplary embodiments.

1. A wearable device for warming a body part of a wearer, the body part having a core part and an extremity part, said wearable device comprising a circulatory system containing a heat transfer fluid, the circulatory system having a first heat transfer element for wearing adjacent the body part extremity for heat transfer from the first heat transfer element to the extremity, and a second heat transfer element for wearing adjacent the body part core for heat transfer from the core to the second heat transfer element, the circulatory system including a pump operable in response to movement of the body part to pump the heat transfer fluid around the circulatory system.

2. A wearable device as claimed in claim 1, the device contained within an insulating fabric housing.

3. A wearable device as claimed in claim 2, the wearable device being an integral part of the housing.

4. A wearable device as claimed in claim 1, the pump having a first chamber having a flexible, relatively inelastic wall material and a second chamber having a flexible relatively elastic wall material, and a passage for one-way flow of fluid from the second chamber to the first chamber.

5. A wearable device as claimed in claim 4, the circulatory system further including a plurality of tubes, the tubes having valves for one-way flow of heat transfer fluid through the tubes, the tubes linking the first chamber to the second chamber, being in fluid communication therewith, and being of a length to extend from the chamber to an extremity of the body part and back.

6. A wearable device as claimed in claim 5, the valves formed integrally with and internal to the tubes.

7. A wearable device as claimed in claim 1, the body part being a hand, the insulating housing being a glove, the pump operable by the wearer making hand movements that are at least one of grasping/ungrasping, wrist cocking/uncocking and finger crooking/uncrooking.

8. A wearable device as claimed in claim 7, the core part being a hand palm and the second heat transfer element located next to the palm when the device is worn.

9. A wearable device as claimed in claim 7, the extremity part being a finger and the first heat transfer element located next to the finger when the device is worn.

10. A wearable device as claimed in claim 1, the body part being a foot, the insulating housing being a boot, the pump operable by the wearer making foot movements that are at least one of walking, articulating one part of the foot relative another part of the foot, articulating the foot relative the ankle, and wriggling the toes.

11. A wearable device as claimed in claim 10, the core being at a bottom surface of the foot and the second heat transfer element located in a sole portion of the boot when the device is worn.

12. A wearable device as claimed in claim 10, the extremity being a toe and the first heat transfer element located next to the toe when the device is worn.

13. A wearable device as claimed in claim 5, tubes of the circulatory system having a range of bore sizes or shapes.

14. A wearable device as claimed in claim 5, tubes of the circulatory system in a “go” portion thereof branching away from the core into tubes of smaller bore to a location to be warmed, the tubes of smaller bore combining in a “return” portion towards the core.

15. A wearable device as claimed in claim 5, portions of the tubes for location next to the body part extremity having shapes configured for high heat transfer.

16. A wearable device as claimed in claim 1, the heat transfer fluid having a freezing point below the freezing point of water.

17. A wearable device as claimed in claim 16, the heat transfer fluid containing ethylene glycol.

18. A wearable device as claimed in claim 1, the pump operable in response to a movement of the body part that is one of (a) an articulating movement, (b) a movement relative to a second body part and (c) a movement involving contact with a reference surface not part of the body.

19. A wearable device as claimed in claim 1, the pump separate from the second heat transfer element.

20. A method of warming a body part using a device worn at the body part, the body part having a core part and an extremity part, the method comprising moving the body part so as to operate a pump forming part of the device to pump heat transfer fluid through a circulatory system forming part of the device, transferring heat from a first heat transfer element forming part of the circulatory system to the extremity part, and transferring heat from the core part to a second heat transfer element forming part of the circulatory system.
